(19) United States
(12) Patent Application Publication
(10) Pub. No.: US 2004/0072298 A1
(43) Pub. Date: Apr. 15, 2004

(54) Stem cell expansion enhancing factor and method of use

(76) Inventors: Guy Sauvageau, Montreal (CA); Richard Keith Humphries, Vancouver (CA)

Correspondence Address: OGILVY RENAULT, 1981 MCGILL COLLEGE AVENUE, SUITE 1600, MONTREAL, QC, H3A2Y3, CA

(21) Appl. No.: 10/680,144

(22) Filed: Oct. 8, 2003

Related U.S. Application Data
(63) Continuation-in-part of application No. 09/785,301, filed on Feb. 20, 2001, now abandoned.
(60) Provisional application No. 60/184,343, filed on Feb. 23, 2000.

(51) Int. Cl. C07K 14/475; C07H 21/04; A61K 38/24; C12N 5/06; C12N 5/08

(52) U.S. Cl. 435/69.1; 435/320.1; 435/354; 435/372; 530/399; 536/23.5

(57) ABSTRACT

The present invention relates to a stem cell expansion factor, and to a method for enhancing hematopoietic stem cell expansion by direct delivery of a protein in the cell and which protein is able to cross cell membrane. The method comprises directly delivering in a HSC an amino acid sequence having the activity of a peptide encoded by a Hoxb4 nucleotide sequence. Once delivered, the amino acid sequence is functionally active in the hematopoietic stem cell and enhances expansion thereof. The amino acid sequence may consist of a HOXB4 protein.

### RELATED APPLICATIONS

The present application is a continuation-in-part of application U.S. Ser. No. 09/785,301 filed on Feb. 20, 2001, which is still pending and which claims the benefit of priority on provisional application U.S. S No. 60/194,343 filed on Feb. 23, 2000, which applications are hereby incorporated by reference.

### BACKGROUND OF THE INVENTION

(a) Field of the Invention

The present invention relates to a stem cell expansion factor, and to a method for enhancing stem cell expansion by direct delivery of a protein in the cell.

(b) Description of Prior Art

Hematopoietic stem cells (HSCs) are rare cells that have been identified in fetal bone marrow, umbilical cord blood, adult bone marrow, and peripheral blood, which are capable of differentiating into each of the myeloerythroid (red blood cells, granulocytes, monocytes), megakaryocyte (platelets) and lymphoid (T-cells, B-cells, and natural killer cells lineages. In addition these cells are long-lived, and are capable of producing additional stem cells, a process termed self-renewal. Stem cells initially undergo commitment to lineage restricted progenitor cells, which can be assayed by their ability to form colonies in semisolid media. Progenitor cells are restricted in their ability to undergo multi-lineage differentiation and have lost their ability to self-renew. Progenitor cells eventually differentiate and mature into each of the functional elements of the blood.

The lifelong maintenance of mature blood cells results from the proliferative activity of a small number of totipotent HSCs that have a high, but perhaps limited, capacity for self-renewal.

The hematopoietic stem cell (HSC) can be operationally defined as a cell responsible for the long-term engraftment of all blood cell types following bone marrow transplantation. Its evaluation should therefore take into account this definition thus implying in vivo testing. There are several assays that have been described to measure the frequency of HSCs. The assay to evaluate stem cell numbers is called the CRU (competitive repopulation unit) assay. This assay combines principles of limiting dilution analysis and competitive repopulation to quantitate HSC frequencies in unknown test populations. In its original description, various numbers of test cells were co-injected with “compromised” helper cells into irradiated (myeloablated) recipients. The helper cells assured short-term hematopoietic reconstitution and are the to be compromised because they have lost most of their long-term repopulating ability as a result of serial transplantation (Mauch, P., Hellman, S. Blood. 74, 872-875, 1989). Because lympho-myeloid elements that originate from the test cell can be identified either by genetic marker or by cell surface antigen (Ly5.1/Ly5.2), it is possible to identify recipients in which a test cell has significantly contributed to long-term repopulation of both lymphoid and myeloid cells (both>1% contribution). The HSC operationally defined by this assay is termed a CRU and its frequency is established based on Poisson statistics from the proportion of mice that meet the repopulation criteria described above. More precisely, the frequency of CRU in the test population is [CRU frequency=1/(No. of bone marrow test cells that repopulated exactly 63% of the irradiated recipients)]. The growing therapeutic use of stem cell transplantation and potential applications of in vitro HSC expansion have focussed attention on defining regulators (both intrinsic and extrinsic) of self-renewal division of HSC.

A variety of in vitro culture conditions have been described that permit substantial expansion of primitive cells detected as long-term culture-initiating cells (LTC-IC) (>50-fold). However, the in vitro expansion of rigorously defined HSC has proven a greater challenge. With careful selection of growth factor combinations and culture conditions, maintenance and even modest but significant net expansion (<10 fold) have been reported for adult mouse bone marrow CRU³⁶ and human cord blood CRU, the latter detected using the NOD/SCID repopulation model. The growth factor requirements appear complex with positive regulators such as FL, SF, and Il-11 being critical, while conversely, certain cytokines such as IL-3 or Il-1 have potentially detrimental effects. CRU expansions so far documented are considerably lower than that observed during the regeneration of CRU following transplantation (in vivo). Additional or alternative stimulatory growth factors (Thrombopoietin (TPO), Steel or bone morphogenetic protein), timely addition of negative regulators to suppress cell cycle and/or novel stromal supports (Moore, K. A. et al., Blood. 89, 4337-4347, 1997) are several promising avenues for achieving increased expansion. Increased understanding of the underlying intrinsic molecular mechanisms regulating HSC growth properties also appears crucial to achieving greater HSC expansion both in vitro and in vitro.

Following bone marrow transplantation (BMT), there is rapid regeneration to normal pre-transplantation levels in the number of hematopoietic progenitors and mature end cells whereas hematopoietic stem cell (HSC) numbers recover to only 5-10% of normal levels. This suggests that HSC are significantly restricted in their self-renewal behavior and hence in their ability to repopulate the host stem cell compartment.

The Hox family of homeobox genes are defined by the presence of a conserved 180 nucleotide sequence called the homeobox. Hox homeobox genes are related by the presence of a conserved 60-amino acid sequence that specifies a helix-turn-helix DNA-binding domain. Increasing evidence points to Hox homeobox genes as playing important lineage-specific roles throughout life in a variety of tissues including the hematopoietic system.

Hematopoiesis is the process by which mature blood cells are continuously generated throughout adult life from a small number of totipotent hematopoietic stem cells (HSC). The HSCs have the key properties of being able to self-renew and to differentiate into mature cells of both lymphoid and myeloid lineages. Although the genetic mechanisms responsible for the control of self-renewal and differentiation outcomes of HSC divisions remain largely unknown, a number of studies have implicated a variety of transcription factors as key regulatory components of these processes.

Among such factors are the mammalian Hox homeobox gene family of transcription factors, consisting of 39 members arranged in 4 clusters (A, B, C and D), initially described as important regulators of pattern formation in a variety of embryonic tissues. These genes are structurally related by the presence of a 183-bp sequence, the homeobox, that encodes a helix-turn-helix DNA binding motif. Apparent stage- and lineage-specific expression of numerous HOXA, B, and C genes has now been demonstrated for both hematopoietic cell lines and primary hematopoietic cells. For example, we have shown that members of the HOXA and HOXB cluster genes are preferentially expressed in the CD34⁺ fraction of human bone marrow cells that contains most if not all of the hematopoietic progenitor cells. Further detailed analysis of Hox gene expression in functionally distinct subpopulations of CD34⁺ cells has shown that genes, primarily located at the 3′ end of the clusters (HOXB3 and HOXB4), are preferentially expressed in the subpopulation containing the most primitive hematopoietic cells

Major new insights into the mechanisms involved in HSC regulation has come from evidence that molecules normally involved in regulating embryonic development also control proliferation and differentiation of hematopoietic cells. Hox genes are part of this family of developmental regulators. Primitive human bone marrow cells express a large number of Hox genes and the expression of these genes decreases as the cells differentiate into more mature elements. Retroviral overexpression of several of these genes assessed in the murine model reveals effects that are specific for each Hox gene tested. For example, Hoxb4 specifically enhances the repopulation potential of HSCs without inducing leukemic transformation. On the other hand, Hoxb3 induces a complete block in the production of CD4⁺CD8⁺ αβ thymocytes but significantly enhances the generation of γδ T-lymphocytes. Hoxa10 inhibits monocytic differentiation but dramatically enhances the generation of megakaryocytic progenitors. It thus appears that each Hox gene, when overexpressed, has the capacity to influence differentiation and proliferation of specific hematopoietic cells and suggest that they each regulate a specific set of target genes.

As most transcription factors, Hox are modular proteins with a DNA-binding domain and a transcriptional activator (or repressor) domain usually located in the N-terminal part of the protein. Most Hox proteins have the small 4-6 amino acid motif required for their interaction with another group of homeodomain-containing proteins called PBX. Hox/PBX cooperatively bind DNA on TGATNNAT sites.

It is known to transduce HSC with a retroviral vector comprising a Hoxb4 gene. For example, in U.S. Pat. No 5,837,507, there is described a gene therapy approach based on the stable integration of a HOX gene in a stem cell, to enhance stem cell expansion. Hematopoietic stem cells (HSCs) genetically engineered to overexpress the Hoxb4 gene have a 20- to 55-fold repopulation advantage over untransduced cells. This capacity of the Hoxb4 gene to selectively enhance HSC regeneration appears to occur without blocking or skewing their differentiation or inducing leukemic transformation. This “Hoxb4 effect” occurs shortly (days) after retroviral transduction and primitive human bone marrow cells can also “respond” to retrovirally engineered Hox gene overexpression. In U.S. Pat. No. 5,837,507, a gene therapy based on the exogenous expression of a HOX gene for the enhanced ability of cells to proliferate to form expanded population of pluripotent stem cell.

Numerous studies have reported that proteins present in the cellular environment can be efficiently transduced into mammalian cells while preserving their functional activity. It was reported that the homeodomain (HD) of a Drosophila Hox gene (Antennapedia or Antp) is capable of translocating across the neuronal membranes and is conveyed to the nuclei. However, the mechanism responsible for this capture remains poorly defined. Interestingly, the Antp protein remains functional once captured by the cell.⁹⁸ It was later demonstrated that this capture of Antp was dependent on a 16-amino acid-long peptide present in the conserved third α-helix of the HD. Comparison between this region of Antp and that of Hoxb4 shows a complete conservation thus suggesting that the Hoxb4 protein could be directly incorporated into the cellular environment where it could be translocated into the nucleus, as observed with Antp.

Intracellular protein delivery was also reported with 2 viral-derived proteins, the HSV VP16 and the HIV TAT proteins. The 86 amino acid HIV TAT protein has been the focus of several studies. TAT is involved in the replication of HIV-1. Several studies have shown that TAT is able to translocate through the plasma membrane and to reach the nucleus where it transactivates the viral genome. It was recently shown that this “translocating activity” of TAT resides within residues 47 to 60 of the protein¹⁰³ and that this 13 mer peptide accumulates in cells (nucleus) extremely rapidly (seconds to minutes) at concentrations as low as 100 nM. The internalization process used by the TAT peptide does not seem to involve an endocytic pathway since no inhibition of uptake was observed at 4° C.

In a recent study, Nagahara et al. have reported the ability of several TAT (11 mer) fusion proteins to be efficiently captured by several cell types (including primary hematopoietic cells). According to a recent communication by these authors, this approach has been used with success with at least 50 different proteins (Nagahara, H. et al., Nat Med. 4, 1449-1452, 1998). The authors have shown that denatured proteins transduce more efficiently than correctly folded proteins. The exact reason for this observation may relate to reduced structural constraints of denatured proteins. Once inside the cells, the denatured proteins are correctly folded by cellular chaperones. The incorporated proteins were shown to preserve functional activity.

In a more recent paper, Dowdy et al. have reported the in vivo (intra-peritoneal) delivery of large (120 kDa) TAT-fusion proteins with a remarkable efficiency of protein transfer to most tissues including “functional protein transfer” to 100% of hematopoietic blood cells in 20 minutes (Schwarze, S. R. et al., Science 285, 1569-1572. 1999). Moreover, the authors showed the absence of toxicity for mice receiving up to 1 mg i.p. of TAT-fusion proteins daily for 14 days.

Autologous and allogeneic transplantation of hematopoietic stem cells using bone marrow or peripheral blood stem cells is a well-established procedure for restoring normal hematopoiesis in patients undergoing ablative treatments for cancer. The major toxicity of allogeneic transplantation is graft vs. host disease caused by immunologic differences between donors and recipients. Current techniques for collecting autologous peripheral blood stem cells require the administration of potentially toxic cytokines and chemotherapeutic agents to the patient to mobilize stem cells from the bone marrow, and subjecting the patient to sometimes multiple leukopheresis procedures to collect a sufficient number of stem cells.

A major limitation in bone marrow transplantation is obtaining enough stem cells to restore blood formation. The overexpression of the Hox4 gene in bone marrow cells using a retroviral vector expands the cells up to 750 fold. However, gene transfer efficiency remains low, and long-term over-expression of the gene could predispose to leukemic transformation.

There is described in U.S. Pat. No. 5,837,507 (issued on November 1998 and wherein one of the co-inventor of the present application is also a co-inventor of this previous patent), a stem cell genetically modified to express exogenous HOXB4 protein. This approach is a gene therapy approach which is not user friendly or clinically feasible. It was not known to the inventors of this US Patent at that time that the HOXB4 protein could cross the cell membrane or that it could be used in a protein therapy for expansion of stem cells.

It would therefore be highly desirable to be provided with a protein therapy (wherein the protein would be able to cross cell membrane) as opposed to a gene therapy for enhancing stem cell expansion in vivo following bone marrow transplantation and/or in vitro prior to the transplantation. Stem cell expansion would permit collection of smaller blood samples, with less discomfort and risks to the patient. It would allow the use of alternative source of stem cells such as those derived from cord blood, for bone marrow transplantation procedures.

### SUMMARY OF THE INVENTION

One aim of the present invention is to provide a protein therapy for enhancing stem cell expansion in vivo following bone marrow transplantation and/or in vitro prior to the transplantation, wherein the protein is able to cross cell membrane. This cellular therapy would be possible by the use of HOXB4 or TAT-HOXB4 proteins as a “stem cell expanding factor”.

In accordance with a broad aspect of the present invention, there is provided a method for enhancing expansion of a stem cell (HSC) population. The method comprises directly delivering to a HSC population an amino acid sequence having the activity of a peptide encoded by a Hoxb4 nucleotide sequence and is capable of crossing cell membrane. Once delivered, the amino acid sequence is functionally active in the stem cell population and enhances expansion thereof.

The amino acid sequence may consist of a Hoxb4 peptide such as the whole Hoxb4 protein or a part thereof.

The amino acid sequence may further comprise an HIV-derived peptide able to cross the cell membrane, such as the NH₃-terminal protein transduction domain (PTD) derived from the HIV TAT protein.

It was surprisingly discovered that HOXB4 protein delivery to hematopoietic stem cells in vitro resulted in enhanced expansion after 4 days.

Alternatively, the protein delivery may be placed under inducible control using a drug inducible system.

In accordance with another broad aspect of the present invention, there is provided a drug-inducible method for enhancing hematopoietic stem cell expansion. The method comprises delivering in a hematopoietic stem cell population a nucleotide sequence linked to a drug-binding protein and encoding one of a DNA-binding domain and a N-terminal domain of a peptide having the activity of a HOXB4 peptide, delivering in the hematopoietic stem cell population a nucleotide sequence encoding the remainder of the DNA-binding domain and N-terminal domains linked to a drug-binding protein, and exposing the hematopoietic stem cell to a dimerizing agent. A functionally active HOXB4 peptide is reconstituted in the hematopoietic stem cell in which are delivered the two nucleotide sequences, thereby enhancing expansion of the hematopoietic stem cell. The binding protein may consist of FKBP12 and the dimerizing agent may consist of FK1012 or an analog thereof.

### BRIEF DESCRIPTION OF THE DRAWINGS

FIG. 1 illustrates the primary structure of HOXB4. HOXB4 is a relatively small protein of 251 amino acids. Based on comparative analysis with paralogs and orthologs, the HOXB4 protein can be divided into 6 distinct domains. A: Foremost N-terminal domain: Conserved from Drosophila to human; B: Very little conservation; proline rich in human Hoxb4; c: Pbx-interacting hexapeptide; highly conserved from Drosophila to human; D: Region between hexapeptide and HD; highly conserved between vertebrate paralogs; E: homeodomain; highly conserved from Drosophila to human.

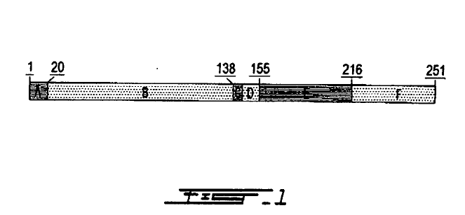

FIG. 2 illustrates results in producing (A), purifying (A and B) and incorporating FITC-labeled TAT-Hoxb4 into hematopoietic cells (C); A: purification of TAT-HOXB4 protein from bacterial lysage; Lane 1: bacterial lysate before purification on Nickel column; Lane 2 and 3: aliquot of TAT-HOXB4 protein after purification (2 different concentrations of Imidazole); B: Western blot analysis of the TAT-HOXB4 protein purified in A; C: FACS analysis of Ba/F3 cells exposed for 20 to 60 minutes to TAT-HOXB4 previously conjugated to FITC and separated from free-FITC by chromatography.

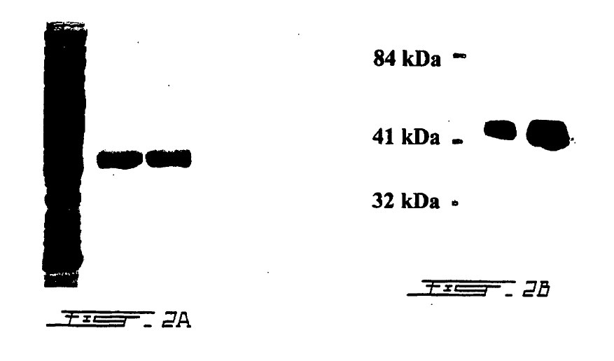

FIG. 3 illustrates increased Human myelopoiesis in NOD/SCID mice transplanted with human CB cells transduced with Hoxa10-GFP compared to GFP control. GFP+CD15+ human cells were measured in recipient mouse BM aspiratees 8 weeks post tx. Circles: individual mice; horizontal line: median number.

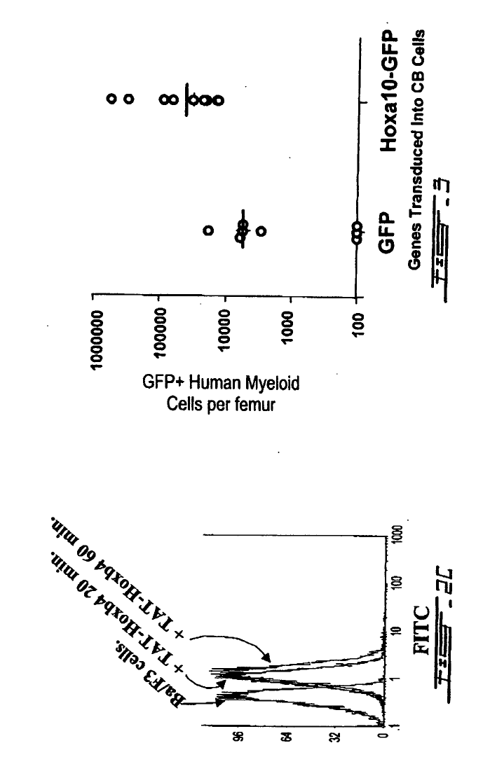

FIG. 4 illustrates (A) the primary structure of the HOXB4 protein divided in 6 different domains; (B) the capacity of mutant HOXB4 proteins to induce proliferative effects in Rat-1 cells or primary bone marrow cells as summarized; The point mutants in C (Try>Gly) and E (Asn>Ser) inhibit the capacity of Hoxb4 to interact with PBX and DNA respectively.

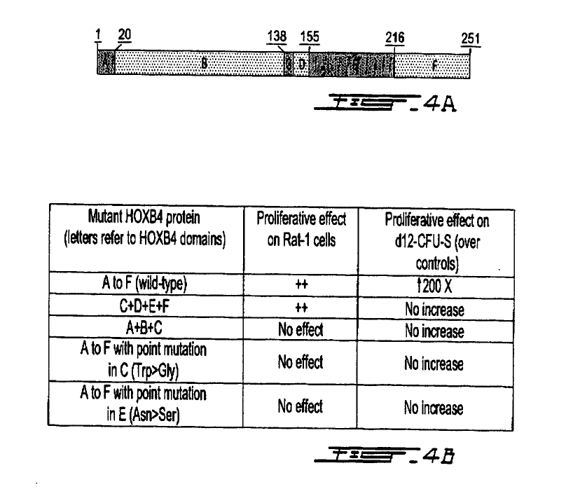

FIG. 5 illustrates a comparison of the domains A and B of the protein (Hoxa4 as SEQ ID NO:1, Hoxc4 as SEQ ID NO:2, Hoxd4 as SEQ ID NO:3, Hoxb4 as SEQ ID NO:4 and Dfd as SEQ ID NO:5).

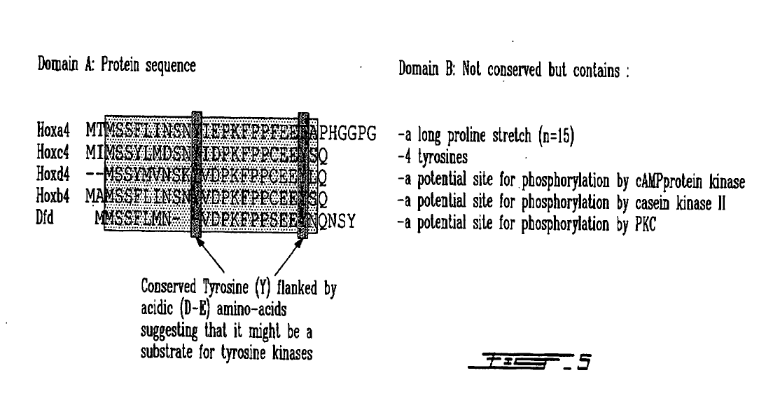

FIG. 6 illustrates a Western blot analysis of nuclear extracts from Rat-1 (lane 1 and 2) and 3T3 cells (lane 3 and 4) transduced with a Hoxb4 (lane 2 and 4) or a neo control (lane 1 and 3) retrovirus.

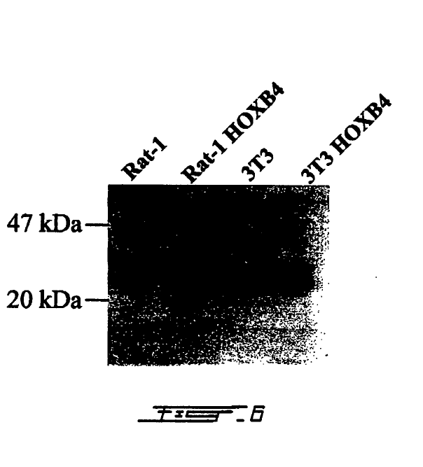

FIG. 7 illustrates Biochemical properties of HOXB4 proteins. a) Schematic representation of TAT-HOXB4 protein. b) Purity of recombinant TAT-HOXB4 as detected on Coomasie blue-stained polyacrylamide gel. BL, bacterial lysate; H, purified TAT-HOXB4. c) HOXB4 levels in 50,000 retrovirally transduced BM cells (lane 8) compared to various concentrations of TAT-HOXB4 (lanes 1-7). d TAT-HOXB4 enters the nuclear of Rat-1 cells. e) Stability of TAT-HOXB4 in medium containing 10% FSC. f) Pulse chase analyses suggesting that t1/2 of intracellular HOXB4 in hemopoietic cells is only ˜1 hr.

FIG. 8 illustrates TAT-HOXB4 promotes in vitro proliferation of bone marrow (BM) cells. a) Experimental protocol used in this study. b) Details of daily schedule of TAT-HOXB4 treatment. c) TAT-HOXB4 promotes the in vitro proliferation of primary BM cells. BSA, bovine serum albumin. d) TAT-HOXB4 enhances the competitive reconstitution potential of cultured BM cells e). Limiting dilution analysis demonstrating that a 4-day exposure to 10 nM TAT-HOXB4 induces HSC expansion. Values shown are expressed based on the input numbers (to) of cells.

FIG. 9 illustrates TAT-HOXB4 stimulates ex vivo expansion of Sca+Lin− cells. a) Increase in total cell numbers (MNC) and myeloid CFC in liquid cultures initiated with sorted Sca−l+Lin− cells and exposed for 4 days to 20 nM TAT-HOXB4 or TAT-GFP. b) TAT-HOXB4 directly promotes the ex vivo expansion of HSCs. Limiting dilution analyses for estimation of HSC frequency were performed as described for FIG. 2e. Results in FIGS. 3a and b represent mean values±SD of 3 experiments (see details in Table 1). c) Lympho-myeloid potential of the ex vivo expanded sca+Lin− cells determined at 16 weeks post-transplant. Representative recipients of −10 or −2 HSCs exposed to TAT-GFP or TAT-HOX4, respectively, are shown. Ly 5.1 cells represented 8% and 60% for the indicated TAT-GFP and TAT-HOXB4-treated cells, respectively. For each sample, 10,000 nucleated cells were analyzed

### DETAILED DESCRIPTION OF THE INVENTION

The term “stem cell” is meant a pluripotent cell capable of self-regeneration when provided to a subject in vivo, and give rise to lineage restricted progenitors, which further differentiate and expand into specific lineages. As used herein, “stem cells” includes hematopoietic cells and may include stem cells of other cell types, such as skin and gut epithelial cells, hepatocytes, and neuronal cells. Stem cells include a population of hematopoietic cells having all of the long-term engrafting potential in vivo. Preferable, the term “stem cells” refers to mammalian hematopoietic stem cells; more preferably, the stem cells are human hematopoietic stem cells.

The term “CRU” means competititve repopulation unit representing long-lived and totipotent stem cells.

Expansion may occur in vitro (prior to transplantation) and/or in vivo (enhanced regeneration of stem cell pools after transplantation.

The expression “direct delivery” is intended to mean delivery of a gene product (i.e, protein) into the cell, as opposed to the insertion of the gene itself in the genome of the cell.

“Protein” is intended to mean any protein which can enhance stem cell expansion and is not limited to the HOXB4 peptide.

“Enhancement” is intended to correspond to substantial self-renewal compared to non-enhanced stem cell expansion.

The protein may be delivered to the hematopoietic stem cell by any means known in the art 2 which results in functional activity of the protein in the cell.

The present invention will be more readily understood by referring to the following examples which are given to illustrate the invention rather than to limit its scope.

### EXAMPLE I

### Hoxb4-Induced Proliferative Effect on Mouse HSC Origin

This example defines the early kinetics, duration and magnitude of Hoxb4-induced enhancement of HSC expansion in the in vivo murine model, determines the requirement for myeloablative conditioning and identifies and optimizes in vitro conditions for achieving Hoxb4 effects on repopulating cells.

Hoxb4 overexpression can significantly increase the rate and level of CRU expansion in vivo, as evident by increased numbers as early as 2 weeks post-transplantation, and ultimate recoveries to normal numbers. Based on these observations, it was hypothesized that Hoxb4 could positively alter HSC self-renewal behavior and that this effect could require conditions existing in myeloablated recipients. It also appears that the “expanding effect” produced by Hoxb4 on the stem cell pool remains subject to mechanisms that normally limit HSC population size, suggesting that expansion potential of the Hoxb4-transduced HSC may be underestimated. These hypotheses were tested by evaluating the kinetics, magnitude and conditions associated with Hoxb4 enhanced mouse stem cell expansion. Proliferation-enhancing effects of Hoxb4 are also manifest in vitro as so far revealed by increased numbers of day 12 CFU-S and competitive growth of transduced cells in short-term liquid culture. Coupled with recent advances in conditions that support CRU self-renewal in vitro and the rapid effect of Hoxb4 seen in vivo, it is shown that Hoxb4 overexpression may potentiate HSC expansion in short-term in vitro culture. This possibility was tested, and in vitro conditions that permit maximal expansion of mouse HSC engineered to overexpress Hoxb4 were identified.

The MSCV-Hoxb4-IRES-GFP or MSCV-IRES-GFP retroviral vectors (henceforth termed Hoxb4-GFP or GFP respectively) were used. No evidence of “promoter shutdown” were seen with the MSCV vector even after repeated transplantations. Thus, GFP expression provides a rigorous indicator of origin from a transduced cell. Donor mice (C57Bl/6J:Pep3b which have the Ly5.1 antigen on the surface of their leukocytes) were injected with 5-Fluorouracil (5-FU, 150 mg/kg) 4 days prior to bone marrow (BM) harvest and infected using a 4 day protocol consisting of 2 days prestimulation in a combination of growth factors (6 ng/ml mIl-3; 100 ng/ml mSF; 10 ng/ml hIl6) followed by exposure to virus-containing supernatants with continued growth factor stimulation on fibronectin-coated dishes for 2 more days with 1 change of media and virus at 24 hours. These infection conditions routinely yielded 40 to 60% gene transfer as monitored by GFP⁺ cells 2 days following termination of the infection procedure.

Transplantation and Kinetics of CRU Regeneration In Vivo

Donor (Ly5.1⁺) BM cells were recovered immediately after the termination of the infection period and transplanted without prior selection at a dose of 2×10⁵ into multiple lethally irradiated recipient mice (C57BL/6J which are Ly5.2⁺). This represented −40 CRU (frequency of −1 in 5,000 in cells immediately after infection (Sauvageau, G. et al., Genes Dev. 9, 1753-1765, 1995) of which 40-60% were transduced (20 transduced CPU per mouse). Aliquots of these cells were maintained in liquid culture for an additional 2 days to assess gene transfer efficiency by FACS analysis for GFP⁺ cells, and plated in methylcellulose culture to monitor the yield and proportion of GFP⁺ colonies (visualized by fluorescence microscopy). Cohorts of recipient mice (3-4 mice per time point) were sacrificed starting at day 4 post-transplant and thereafter at days 8, 12, and 16 and then week 4, 6 and 8 to measure donor-derived contributions to bone marrow cellularity, clonogenic progenitors and CRU content These time points were chosen in order to define the very early kinetics of CRU reconstitution not previously assessed, and to better define the earliest time at which plateau CRU levels are reached. CRU measurements were carried out by limiting dilution analysis of secondary transplant recipients. Four months following transplantation, blood samples were obtained from CRU assay (secondary) recipients and analyzed by FACS for evidence of significant (>1% lymphoid and 1% myeloid) contribution from transduced (GFP⁺ Ly5.1⁺) or non-transduced (GFP⁻ Ly5.1⁺) cells in the initial donor mouse. CRU frequencies in the original donor mice were then calculated.

Determinations were repeated at 6 months post-transplant to verify the long-term repopulating ability of the CRU measured. At this time, secondary assay recipients were sacrificed and donor contributions confirmed by FACS analysis of thymus and bone marrow (BM) and clonal assessment of provirally-marked CRU carried out by Southern blot analysis of proviral integration patterns. Using unsorted cells in the initial transplant allowed to assess contributions to reconstitution of the various hematopoietic compartments in primary and secondary (CRU assay) mice by monitoring for the presence (or absence) of GFP⁺ expression and the donor-specific cell surface marker Ly5.1 thus providing an additional control for documenting Hoxb4 effects. In recipients of Hoxb4-infected BM, there were essentially exclusive (>95%) reconstitution of primary mice with transduced cells (evident by high proportion of GFP⁺ progenitors, BM cells, etc.) and of CRU (evident by the presence of GFP⁺ donor-derived cells in CRU assay recipients even at limiting dilution). Together these experiments provide important new data relating to the kinetics and duration of Hoxb4 effects on CRU regeneration and help guide further studies to optimize and extend this effect.

Estimating the Maximal Expansion (Self-Renewal) Potential of Hoxb4-Transduced CRU by Serial Transplantation Analyses

In the absence of optimized in vitro conditions for maximal CRU expansion, the in vivo environment was relied upon in order to determine the maximal expansion of a given CRU (Hoxb4-transduced or not). Normal (or neo-transduced) BM CRU can expand by ˜20-fold in vivo following BMT into myeloablated mice. In sharp contrast, Hoxb4-transduced CRU expanded by ˜900-fold under the same conditions. These numbers are derived from mice reconstituted with 10 to 40 CRUs and therefore do not necessarily reflect the expansion per individual CRU, but rather for the whole population of CRU.

To measure the maximal in vivo expansion of individual Hoxb4-transduced CRU, numerous lethally irradiated recipients were reconstituted with limiting numbers of Hoxb4-transduced CRU. Six months after BMT (long-term reconstitution), recipients of 1 CRU (limit dilution) were sacrificed and CRU expansion measured as described above. CRU determination were performed on 10 different primary recipients of 1 Hoxb4-transduced CRU (expansion of 10 different Hoxb4-transduced CRU were measured). This experiment provides information on the possible heterogeneity of the Hoxb4 effect, if there is—equal expansion of each CRU or preferential expansion of a subgroup of cells. These experiments were repeated over the course of at least 3 serial transplantations. Together these studies reveal the self-renewal capacity of individual CRU (monitored by clonal analysis) and provide valuable information about the intriguing possibility that Hoxb4-transduced CRU have an unlimited self-renewal capacity.

To minimize “dilution effects”²⁸ as a trivial cause for a decline in CRU number, the transplant dose used for the first and subsequent serial transplants were adjusted to ensure the presence of at least 1 CRU in the bone marrow innoculum (measured by CRU assay). For example, each serial transplant resulting in at least a return to 10% of normal levels represents a net expansion in (Hoxb4-transduced) CRU numbers of 2000-fold (input=1; output=10%×20000 CRU per normal mouse or 2000 CRU).

Selected secondary (tertiary, etc.) recipients transplanted with one Hoxb4-transduced CRU were followed for extended times post-transplant to verify the long-term repopulating nature of the CRU detected and to assess whether there is any decline in the “quality” of serially transplanted CRU as indicated by decreased levels of lymphoid and/or myeloid reconstitution in these recipients. For all of the experiments described, parallel experiments were also conducted with control-GFP transduced BM cells. In order to draw definitive conclusions on the “quality” of a given CRU, clonal analysis (persistence of proviral integration patterns) were also performed on secondary and tertiary recipients.¹⁵ These experiments provide a unique opportunity to define the potential for (Hoxb4-transduced) HSC expansion and a benchmark for attempts to achieve similar in vitro expansion.

In Vivo Conditioning Requirements for Hoxb4 Effects

In the setting of total myeloablation, CRU levels rapidly rise during the early transplant period but plateau at normal levels along with full hematopoietic recovery of the recipient. These findings suggest that conditions established during myeloablation may be a requisite for the observed Hoxb4 effects in vivo. To test this, hematopoietic contributions of Hoxb4-GFP were monitored versus normal (transduced and not) BM cells following transplant of untreated or minimally ablated recipients achieved by low dose irradiation. The experimental conditions were modeled after those described by Quesenberry et al. which have shown significant (up to 40%) contributions to hemopoiesis by donor cells transplanted at very high cell numbers (a total of 2×10⁸ marrow cells over 5 consecutive days) into untreated recipients or at modest numbers (a single infusion of 10⁷) into mice receiving low dose sub-lethal irradiation (100 cGy). Rapid cell cycle such as associated with 5-FU treated BM may significantly compromise hematopoietic contributions in non-ablative settings⁷⁸. Moreover, relatively large numbers of cells are required. To circumvent both potential problems, BM was harvested from mice previously transplanted (with Hoxb4-transduced cells) under standard ablative conditions 3-4 months earlier and when it was expected they had recovered to normal CRU levels. In initial experiments, 10⁷ BM cells from such a Hoxb4 transplant recipient or an equivalent number from unmanipulated normal mice were transplanted into recipients that were untreated, had received minimal irradiation (50 or 100 cGY) or had total myeloablation (900 cGy), and donor engraftment was monitored by sampling peripheral blood for Hoxb4 transduced cells (GFP⁺) or normal BM-derived (Ly5.1⁺) cells. Transgenic mice (n=2 lines, backrossed 9 times into C57Bl/6J background) that express Hoxb4 in hematopoietic cells were generated. Whether these mice express the transgene in Sca1⁺lin⁻ BM cells and whether the proliferative activity of Hoxb4 on CRU is present in these mice may be evaluated. If so, the Hoxb4 transgenic mice may be used as a source of donor cells.

Significant hematopoietic contributions by normal cells at these modest transplant cell doses is only expected with partial (100 cGy) or complete ablation. Hoxb4 BM transplantation may have several different outcomes each having interesting interpretations. Results equivalent to that seen for normal marrow argue that the Hoxb4 effect requires stimuli triggered by a degree of myeloablation and regenerative stress. This may be further examined by tests over a broader range of irradiation doses (350 cGy, 600 cGy) to see if increased Hoxb4 BM contributions can be achieved at non lethal irradiation doses. Greater contributions for Hoxb4-overexpressing cells compared to normal controls with minimal ablation (50 and/or 100 cGy) but not in the absence of conditioning would be consistent with a need for moderate stem cell ablation and possibly additional stimuli present with low dose irradiation. Significant Hoxb4 cell contributions in unconditioned host provides novel evidence of the competitive growth advantage of Hoxb4 transduced cells and argues that it can occur under “homeostatic” conditions.

It is conceivable that in the absence of myeloablation, it may take longer for Hoxb4-transduced cells to “outcompete” or that some additional stress needs to be imposed. This may be explored by prolonged observation and treatment of mice with cytotoxic drugs such as 5-FU. To further test the possibility that growth factors triggered during hematopoietic regeneration play a role in the Hoxb4 effect, the effect of growth factor administration during the early transplant period (first 2 weeks) was tested under all transplant conditions (untreated, low dose and lethal irradiation). Initial candidates included SF and IL-11, based on results from Iscove²⁶ suggesting that these could enhance regeneration of normal BM and evidence of their potent effects on hematopoietic expansion in vitro. Depending on the lack or presence of effects, additional growth factors were tested e.g., IL-3, FL and TPO. For additional clues to the possible factors involved, mice set up for the kinetic analyses of regeneration were used to monitor, by ELISA assay, serum levels of these candidate growth factors in the early post transplant period. These studies provide important insights into critical determinants of Hoxb4 effects on HSC growth.

In Vitro Expansion of Hoxb4-Overexpressing CRU

In a pilot study, CRU numbers were measured at >10-fold above input values in cultures initiated with Hoxb4-transduced cells and maintained for 4 days in vitro after viral transduction using conditions described above. This initial data suggests that Hoxb4 has the capacity to induce significant CRU expansion in vitro (if cells are maintained in culture for at least 4 days post-transduction). One major goal of these studies was to determine optimal conditions for Hoxb4-enhanced CRU expansion in vitro. Day 4 5-FU BM from C57Bl/6J:Pep3b (Ly5.1⁺) donors were infected with Hoxb4-GFP or GFP retrovirus as mentioned above. Immediately after the infection period GFP⁺ BM cells were isolated by FACS and assayed for clonogenic progenitors, day 12 CFU-S and CRU content. Aliquots were then placed in replicate liquid culture under various conditions and changes in total cellularity, progenitor (CFC and day 12 CFU-S) and CRU content determined at 2 day intervals initially up to a total duration of 14 days. To determine whether accessory cells (macrophages, etc.) are required, parallel experiments were performed with purified GFP⁺Scal⁺lin⁻ BM cells.

Experiments were initially conducted with non-sorted cells (mixture of transduced and untransduced cells). The growth of Hoxb4-transduced cells including CRU was compared to the nontranaduced cells in the same culture and to the control cultures established with mixtures of GFP and non-transduced cells Initial conditions chosen were modeled after those shown to support at least modest increases in CRU numbers for normal BM (FL, SF and IL-11 in serum free medium). Additional growth factors were also tested alone and in combination using a factorial design method for optimizing conditions for in vitro expansion of primitive murine and human hematopoietic stem cells. Interesting additional candidate factors tested include thrombopoietin (TPO) based on studies indicating its potential to enhance stem cell recovery in vitro. Confirmation of CRU expansion suggested by net increases in CRU number over input was sought by analysis of proviral marking to detect common patterns in multiple recipients of cells from the same culture to document CRU self-renewal in stromal LTC. If significant CRU expansions was apparent, this effect was further assessed by establishment of replica cultures initiated with individual GFP⁺Scal⁺lin⁻ BM cells which were then individually monitored for cell division and CRU output at a clonal level.

### EXAMPLE II

These studies were extended for the first time to both in vitro and in vivo models of human hemopoiesis, to evaluate in human hematopoietic cells, the effect of Hoxb4 overexpression on the in vitro and in vivo expansion of primitive long-term repopulating cells assayed in the immuno-deficient (NOD/SCID) mouse model.

Given the long established methods for efficient genetic manipulation and rigorous quantitative measures of murine HSC, functional studies of Hoxb4 have so far concentrated on murine BM cells. The recent development of assays for primitive human repopulating cells based on the immuno-deficient mouse model and improved conditions for gene transfer to NOD/SCID CRU now present an opportune time to extend investigations directly to human cells. Studies of Hoxa10 overexpression on growth of transduced human cord blood cells both in vitro and in vivo were recently carried out. Key findings include marked increases in “replating” ability of Hoxa10-transduced CFC, increased nucleated cell expansion (with a skew to blast cell production) in serum-free liquid culture and, most strikingly, greatly enhanced myelopoiesis in NOD/SCID mice.

These findings are remarkably similar to the effects of Hoxa10 overexpression in the murine model and support the hypothesis that Hox gene overexpression could impact on human hematopoietic cell growth, and encourage a direct test of the ability of Hoxb4 to influence primitive human hematopoietic cell growth potential.

The experiments were modeled from murine studies. High titer viral producers (>5×10⁵) were generated for the control GFP vector in the PG13 packaging line generated PG13 producers for Hoxb4-GFP virus. Infections of cord blood (CB) cells enriched for CD34⁺ cells by lineage depletion (using StemSep columns) were carried out using optimized conditions that were established to achieve in excess of 40% gene transfer with the GFP virus to human LTC-IC and at least 10-20% to NOD/SCID CRU. Equivalent gene transfer to CRU from adult BM is possible. Lenti-based vectors were also evaluated and may be employed if their early promise of affording high gene transfer and increased stem cell recovery without prolonged in vitro culture are realized. Possible effects of Hoxb4 overexpression may first be assessed with relatively straightforward in vitro methods. To minimize the scale of experiments involving costly serum free reagents and growth factors, transduced primitive cells may be pre-enriched by FACS isolation of CD34⁺CD38⁻GFP⁺ cells 1 to 2 days after termination of the infection procedure. Starting clonogenic progenitor content may be assessed using methylcellulose assay and the “replating” capacity of these resulting colonies compared for Hoxb4- and GFP-control transduced cells. The initial LTC-IC content may be assessed by limiting dilution assay and the progenitor output per LTC-IC determined after 6 weeks in culture as another possible measure of a Hoxb4 effect on primitive cell growth.

Serum-free liquid cultures with selected growth factors may also be established and yield of phenotypically defined subsets (CD34⁺CD38⁻, total CD34⁺, total nucleated cells) monitored over 1 to 2′ weeks, as well as output of clonogenic progenitors and LTC-IC. Initial culture conditions chosen may be those previously documented to support significant expansion of both LTC-IC and CRU (FL, SF, IL-3, IL-6 and G-CSF). Additional factors (TPO, etc.) may be tested using factorial design experiments. If positive effects of Hoxb4 are detected with any or all of the above assays, they may be tested directly on expansion of CRU using the limiting dilution assay in NOD/SCID. The low starting frequency of CRU in cord blood (−6 per 10⁵ CD34⁺ cells, or some 100 fold lower than LTC-IC) dictates considerably larger scale experiments and thus cultures may be initiated with cells recovered after infection of CD34⁺lin⁻ CB cells without further enrichment to avoid excessive sorting times. The presence of the GFP marker may enable direct tracking of transduced CRU versus non transduced CRU repopulation in recipient mice. Current optimized conditions support ˜5-10-fold expansion of normal CB NOD/SCID CRU in 1 week serum-free liquid culture conditions. If increases in this are seen following Hoxb4 transduction, the potential duration of expansion and effects of other growth factor combinations and levels may be explored in a manner similar to that outlined for the murine studies.

The human CRU assay has reached a state of refinement in which it has been possible to additionally demonstrate CRU regeneration in primary NOD/SCID recipients by carrying out a CRU assay in secondary recipients in a manner identical to that employed in the murine system (Sauvageau, G. et al., Genes Dev. 9, 1753-1765, 1995; Thorsteinsdottir, U. et al., Blood. 94(8), 2605-2612, 1999). Accordingly, cord blood transduced with the Hoxb4-GFP retrovirus (or Lentiviral vector when available) may be transplanted into NOD/SCID recipients and 6-8 weeks post-transplant mice sacrificed for measure of CRU numbers using limiting dilution assay in secondary recipients. Levels of regeneration may be compared to those achievable with unmanipulated cord blood and control GFP transduced cord blood. Additionally, whether growth factor administration (SF, IL-3, GM-CSF and Epo 3×wk. for last 2 wks. before sacrifice) during the repopulating phase is either necessary or can enhance Hoxb4 effects may be explored. These studies may be further extended to analysis of CRU expansion from adult sources.

Together, these studies provide new insights into the potential and conditions for HSC expansion and help to identify and characterize mediators of the Hoxb4 effect and harnessing it through alternative methods to achieve the effect by transient exposure to Hoxb4 (adenoviral or protein based) or drug-inducible expression systems.

### EXAMPLE III

### Identification of the Minimal Domain(s) of the HOX4 Protein Necessary to Regulate Expansion of HSCs

Rat-1 fibroblasts overexpressing Hoxb4 proliferate in low concentrations of serum, show a reduction in G_{I} phase of the cell cycle and can form colonies in soft agar (so-called anchorage independent growth). A structure-function study was performed to identify region(s) of the HOXB4 protein that may be important for these effects. The results from these experiments suggest that both the DNA-binding and the PBX-interacting domains of the HOXB4 protein are necessary. The NH₂-terminal region of the protein seemed, however, dispensable for the effect of Hoxb4 on Rat-1 cells.

Preliminary experiments performed with BM cells indicate that the NH₂-terminal region of Hoxb4 is required for the enhanced expansion in Hoxb4-transduced primitive bone marrow cells. This suggests that Hoxb4-induced proliferation of certain types of hematopoietic cells may involve the NH₂-terminal region of Hoxb4 in addition to the DNA-binding homeodomain and the PBX-interaction motif.

Construction of Mutants

The experimental procedures for these studies parallel those described above (retroviral gene transfer to primary bone marrow cells). The Hoxb4 mutants may be overexpressed in mouse bone marrow (BM) cells and quantification of the effects produced by these mutant forms may be measured using the CRU assay. The “CRU-expanding activity” of the N-terminal deletion mutant was tested and compared to that of full-length Hoxb4. The results from this experiment (n=2 mice only) clearly indicated that CRU numbers were increased to pre-transplantation levels for Hoxb4-transduced cells whereas CRU numbers were similar to neo-controls (reduced by ˜30-fold) in recipients of bone marrow cells transduced with the N-terminal deletion mutant (domain C to F mutant of Hoxb4). This clearly indicated that this N-terminal domain is necessary for the proliferative activity of Hoxb4 on HSC.

In order to define the minimal “active” region in the N-terminal domain of Hoxb4, we sought for conserved subdomains within this region were sought for by comparing the amino acid sequence between insect Hoxb4 (Deformed, Dfd) to that of the other Hox gene products of the 4^{th} paralog derived from various species (Hoxa4, Hoxd4 and Hoxc4) . 2 domains were identified (A and B). Domain A (amino acid 3 to 23 of Hoxb4) contains 20 highly conserved (from insect to human) amino acids which include two conserved tyrosine residues that are flanked by acidic residues, suggesting that these motives may represent substrates for tyrosine-related kinases. Domain B is poorly conserved but contains a praline stretch and several potential serine/theronine residues, one of which is a consensus site for casein kinase II (CKII), a kinase recently shown to associate and modulate the function of insect Hox proteins.

Hoxb4 mutants lacking domain A alone or domain B alone (A+C+D+E+F) were generated and tested as indicated above. In addition, 3 point mutants which include the two tyrosine residues in domain A and the site for CKII in domain B were generated and tested at the same time because the readout for these experiments (CRU assay) was too long. Prior to making these tyrosine “mutants” (Y>F), whether any of the tyrosine residues in Hoxb4 are phosphorylated in vivo were evaluated. To do this, the anti-phosphotyrosine 4G10 antibody was used on HOXB4 protein immuno-precipitated from different hematopoietic cell lines (K562 and FDC-P1 cells) and in Rat-1 cells engineered to overexpress Hoxb4. Finally, a mutant lacking the proline-rich region (amino acid # 61 to 79) was constructed and tested.

Prior to bone marrow transduction experiments, each mutant was tested in Rat-1 fibroblast in order to determine whether a nuclear protein of the expected size is produced using western blot analysis. If not, a nuclear localization sequence (NLS) derived from c-myc was added. An antibody to both the N-terminal and C-terminal domains of Hoxb4 (VA Medical Center, USF, California) was used to detect HOXB4 proteins in Rat-1 cells.

Once the minimal domain(s) of Hoxb4 that are required for CRU expansion are know, Hoxb4-interacting proteins may be isolated by using a yeast-two-hybrid screen. Alternatively, depending on the results obtained (the serine mutant for CKII binding is dysfunctional), the importance of candidate protein partners may be tested (CKII in this example).

### EXAMPLE IV

### Identification of Effectors of Hoxb4-Induced Proliferative Effects

This example uses an approach similar to a yeast-two-hybrid screen to isolate a novel interacting partner to PBX1 from a cDNA library prepared from human fetal liver cells at a time of active hemopoiesis to isolate Hoxb4-interacting protein(s) to identify proteins that specifically interact with Hoxb4.

Preliminary studies with various Hoxb4 mutant constructs have suggested that both the DNA-binding and Pbx-interaction motives of Hoxb4 are required for its proliferative activity on Rat-1 fibroblasts and day 12 CFU-S cells (and thus likely on CRU). The N-terminal domain of the protein is also required for its activity in primary bone marrow cells (d12 CFU-S and CRU). Since PBX1 (a Hoxb4 DNA-binding co-factor) interacts with the conserved hexapeptide and homeodomain and since primitive bone marrow cells express PBX1 (also PBX2 and 3), a screen for Hoxb4-interacting proteins could exclude these 2 domains (high likelihood of picking up PBX which has been shown to interact with other Hox proteins in yeast-two-hybrid screens and which appears to be required for the proliferative activity of Hoxb4 on Rat-1 cells).

The specific requirement of the N-terminal domain of Hoxb4 for the proliferation of hematopoietic cells (but not for Rat-1 fibroblasts) suggests the presence of a unique co-factor in hematopoietic cells. The goal of this example is to isolate a protein partner to this N-terminal region of Hoxb4.

Yeast-two-hybrid systems are based on the “conditional expression of a nutritional reporter gene (HIS3 or LacZ) to screen large numbers of yeast transformed with a specially constructed fusion library for interacting proteins”. This conditional expression of reporter genes is induced by the in vivo reconstitution of a functional Gal4 transcription factor resulting from the interaction between two fusion proteins (one which contains the DNA-binding domain (DBD) and, the other, the activation domain (AD) of Gal4). In this case, a fusion protein between Hoxb4 (specific subdomains of the N-terminal region depending on the results of the previous section) and the DBD of Gal4 (Hoxb4-Gal4^{DBD} would be used to screen for a Hoxb4-interacting protein fused as an expression library to the AD domain of Gal4.

Once a partner to Hoxb4 is identified, its capacity to specifically interact with Hoxb4 may be demonstrated. To this end, this new protein may be tagged (HA, MYC and FLAG tags and antibodies are currently in our possession) and co-immuno-precipitation studies and mammalian two hybrids may be performed to determine whether this protein is part of a protein complex with Hoxb4.

cDNA Library

The Matchmaker Gal4 two-hybrid system III (Clontech) may be used. A series of expression libraries fused to the cDNA encoding the activation domain of Gal4 (herein called “library protein AD”) are commercially available. A library made from E14.5dpc mouse fetal liver may be used because fetal livers of that age contain significant numbers of HSC.

To Engineer a Functional TAT-HOXB4 Protein and Test the Incorporation and Persistence (Half-Life) of this Protein in Primitive Hematopoietic Cells

Using the pTAT-HA plasmid developed by Nagahara et al. (1998), we will subclone a full-length Hoxb4 cDNA in frame and downstream to the His6-TAT-HA tag. The protein will be produced in bacteria and purified exactly as described by Nagahara (1998).

The specificity of interaction between Hoxb4 and the identified partner(s) may be tested using standard co-immunoprecipitation assays and mammalian two hybrid system. Direct interaction between the 2 proteins may then be determined using classical pull down experiments. Whether this partner alters the DNA-binding specificity of the Hoxb4 (or Hoxb4-PBX) may also be investigated using EMSA studies. Finally, the involvement of this protein in mediating the proliferative effect of Hoxb4 on CRU may be tested using functional biological studies (retroviral gene transfer, knock out, etc.).

### EXAMPLE V

### Approaches to Achieve Enhanced HSC Expansion Based on Transient Exposure to Hoxb4

The effect of Hoxb4 on CRU expansion appears to occur very early (days) after retroviral gene transfer. Transient (approx. 1-2 wk.) gene transfer into primitive bone marrow cells can be achieved with high efficiency using adenoviral vectors and possibly with TAT-fusion proteins which allow the direct uptake of extracellular proteins into most cell types tested to date (including HSC). HSC which transiently express Hoxb4 (by either adenoviral gene transfer or by exposure to TAT-HOXB4 fusion protein) may benefit from the same repopulation advantage observed with HSC engineered by retroviral gene transfer to overexpress Hoxb4. This experiment tests the feasibility of this approach using the HOXB4 protein as a stem cell expanding factor.

Transient Expression of Hoxb4 in Primitive Bone Marrow Cells Using Adenoviral Gene Transfer

Conditions for high efficiency adenoviral gene transfer to primitive bone marrow cells have recently been defined. Hoxb4 adenoviral vectors may be produced to effect adenoviral gene transfer to primitive mouse and human bone marrow cells using a high titer adenovirus encoding the bacterial β-galactosidase gene. If quiescent freshly isolated Scal⁺Lin⁻ bone marrow cells can not be infected with this β-galactosidase virus (MOI of 200), an infection efficiency of 45-60% of the same cells exposed for 2-3 days to IL-3 (6 ng/ml), IL-6 (10 ng/ml) and steel (100 ng/ml) may be obtained.

Transduction of Proteins into Mammalian Cells

It was surprisingly discovered that most of the Hoxb4 stem cell expanding effect was present at 2 weeks post transplantation (and possibly earlier). It was also surprisingly discovered that TAT-HOXB4 protein delivery to stem cells could be done in vitro before bone marrow transplantation and also in vivo during the early phase of reconstitution if required.

Use of TAT-GFP and TAT-Hoxb4 to Determine whether Primitive Mouse and Human Bone Marrow (BM) Cells have the Capacity to Uptake TAT-Fusion Proteins

TAT-GFP and TAT-HOXB4 proteins were generated and purified. Results show that these proteins are readily incorporated in a dose-dependent manner into Ba/F3 cells with maximal uptake at 60 minutes.

The following experiment determines whether primitive BM cells (Scal⁺Lin⁻) can also uptake these proteins. This may be measured using FACS analysis. The intensity of protein uptake in Scal⁺Lin⁻ cells may be compared to that of mature mononuclear (lin⁺) BM cells. Similarly, primitive human BM cells (CD34⁺CD38⁻ and CD34⁻Lin⁻) may be tested for their capacity to incorporate TAT-GFP and TAT-Hoxb4. The concentration of TAT-proteins to be tested may vary between 10 to 500 nM as reported by Nagahara et al. (1998).

Once studies with TAT-GFP and TAT-Hoxb4 are optimized (protein transfer to primitive bone marrow cells), the internalized TAT-HOXB4 protein as being localized in the nucleus and functional may be demonstrated.

Once optimal conditions are defined with TAT-Hoxb4-FITC, cells may be exposed to non-FITC HOXB4 (TAT- or not) proteins and western blot analysis may be done on cellular extracts (both nuclear and cytoplasmic) at various time points in order to estimate the half-life of the incorporated proteins. The protein levels obtained may be compared to those normally achieved with cells transduced with “Hoxb4 expressing retrovirus”, to adjust the dose of protein necessary to mimic the effect observed with cells engineered to overexpress Hoxb4 using retroviral gene transfer. With these data, the functional capacity of this HOXB4 protein may be tested.

As mentioned above, the HOXB4 protein may have the inherent capacity to penetrate through the cytoplasmic membrane. This may obviate the need for the TAT fusion peptide. In a parallel experiment, a His-tag HOXB4 protein may be produced (without a TAT). For these, the PET24 vector may be used. Briefly, Hoxb4 cDNA may be subcloned in frame with the His-tag in PET24 using standard procedures. Once subcloning is finished (in DH5), the plasmid is then transferred in BL21 bacteria for protein production. The recombinant protein is then purified such as on a nickel column.

Biological Activity of the Fusion TAT-Hoxb4 or the HOXB4 Protein Using a Quick Screening in Vitro Culture System where Hoxb4 was Previously Reported to Exert a 200-500 Fold Effect in Less than 7 Days (Delta CFU-S Assay)

The biological activity of the recombinant (TAT-HOXB4 or His-HOXB4) proteins may be tested first using a surrogate assay, the delta CFU-S assay, as described previously. In this assay, it is possible to directly test in 19 days (7 days of in vitro culture+12 days of in vivo assay) whether a protein is functional. In these experiments, cells may be exposed during the 7 day culture to a concentration of TAT-HOXB4 protein which allows equal or higher levels of intracellular Hoxb4 molecules than achieved with retroviral gene transfer.

Capacity of TAT-HOXB4 Protein to Induce Expansion of Mouse and Human HSC

In the event that CFU-S expansion is achieved with the recombinant HOXB4 proteins, CRU expansion may be tested. In these experiments, the TAT-HOXB4 or the His-HOXB4 recombinant protein may be added to cultures of mouse bone marrow (BM) cells exposed 4 days earlier to 150 mg/kg of 5-FU (in vivo) and prestimulated in vitro for 2 days in the presence of growth factors (IL-3, IL-6 and steel) as mentioned above for retrovirally-transduced cells. The cells may then be exposed to “optimal” concentrations of the TAT-HOXB4 protein during 4 days in medium which includes the growth factors mentioned above. Longer periods of exposure to HOXB4 protein may also be obtained by in vivo administration of the protein (TAT-HOXB4) as recently described by Schwarze et al. (Schwarze, S. R. et al., Science 285, 1569-1572. 1999).

Once optimization is achieved with mouse bone marrow cells, these experiments may be repeated with human (cord blood CD34⁺lin⁻CD38⁻) cells that are injected into NOD/SCID mice at limiting dilution to measure CRU.

This experiment used adenoviral gene transfer and direct protein delivery to test the possibility that Hoxb4 or TAT-Hoxb4 represents a genuine stem cell expanding factor.

### EXAMPLE VI

### Development of a Dominant, Drug-Inducible System for Hoxb4 Enhanced HSC Expansion

Hox proteins are highly modular with well-recognized DNA-binding homeodomain (HD) and PBX-interacting hexapeptide flanking this HD. The Hox-PBX-DNA interaction was recently solved by crystallography where it was shown that the N-terminal region of Hox proteins is dispensable for DNA-binding activity. Using principles extensively exploited in the mammalian two hybrid system, a Hoxb4 DNA-binding domain (mutant C−F) and Hoxb4 N-terminal domain (mutant A+B) were expressed, each linked to the FK506 binding protein (FKBP12) in mouse primary bone marrow cells. These hybrid proteins thereafter called [FKBP-Hoxb4 C−F] and [FKBP-Hoxb4 A+B] respectively, can undergo in vivo dimerization via the intracellular “dimerizing” agent FK1012 to generate a functional HOXB4 protein.

FKBP12 as a Dimerization Partner

The most studied system for inducible heterologous dimerization of fusion proteins is the rapamycin FKBP-FRAP (FKBP-rapamycin binding protein). In this system solved by crystallography, the immunosuppressant rapamycin binds to both FKBP and FRAP fusion proteins thereby reconstituting a functional protein. This has been tested with numerous fusion proteins and shown to be very effective. However, in contrast to FK506, rapamycin was shown to be an effective inhibitor of cell cycle progression. However, this property is incompatible since Hoxb4 induces expansion and thus proliferation of CRU. Recent studies have reported a new rapamycin derivative which still effectively binds to FKBP12 but with very little anti-proliferative and immunosuppressive activity.¹⁰⁸ Other versions of rapamycin with similar properties may also be used.

Another well described system may be used, the FK1012-FKBP. FK1012, a dimeric form of FK506, efficiently dimerizes FKBP12 and does not alter cellular proliferation (Clackson, T. et al., Proc Natl Acad Sci USA. 95, 10437-10442, 1998) This system (FKBP12 plasmids and FK1012 analog AP20187) has been used to reconstitute, in a dose-dependent fashion, the activity of transcription factors including GAL4 (DBD)-VP16 (transactivation domain) heterologous transcription factor on a reporter system using skin keratinocytes and fibroblasts. The synthetic AP20187 compound is more potent than FK1012 and is very similar to AP1903.

Use of Retroviral Vectors to Express Both [FKBP12-Hoxb4 A+BP] and [FKBP12-Hoxb4 C−F] Products

The structure-function studies performed with Hoxb4 clearly showed that the complementary N- and C-terminal mutants of Hoxb4 are dysfunctional (no expansion of d12 CFU-S). A functional HOXB4 protein may be reconstituted in vivo using retroviral gene transfer and the FPBP-Hoxb4 fusion constructs mentioned in the previous paragraph. For these studies, [FKBP-Hoxb4 C−F] and [FKBP-Hoxb4 A+B] cDNAs may be introduced downstream to the retroviral LTR thus generating 2 different retroviruses with 2 distinct markers for selection (GFP and YFP for [FKBP-Hoxb4 C−F] and [FKBP-Hoxb4 A+B], respectively). Following retroviral gene transfer, transduced bone marrow cells may be sorted based on GFP and YFP expression and tested, in the presence of AP20187, to induce CRU expansion. Cells transduced with each retrovirus alone and the combination of both may be tested in parallel experiments. With VSV virus, “double-gene transfer” to mouse BM cells may be obtained in the range of 50%. After sorting, the cells may be tested first for CFU-S activity and, if functional, in CPU assays as described above. These experiments generate a drug-inducible system to build a model for dominant clonal selection of transduced HSC.

Before functionally testing the reconstituted Hoxb4 partners in vivo, whether the 2 proteins dimerize in the presence of AP20187 (in hematopoietic cells lines) may be tested by electromobility gel shift (EMSA). This may be done by incubating the cellular lysates (from cells treated or not with AP20187) with an antibody specific to the N-terminal (non DNA-binding) domain. The presence of a supershifted large complex would be the signature for hetero-dimerization between the carboxy (domains C−F) and the amino-terminal (domains A+B) region of Hoxb4.

There is a potential problem for homodimers to functionally interfere with the reconstituted full-length (heterodimerized) Hoxb4. Co-expression of deletion mutants together with (full-length) Hoxb4 may be tested to ensure that none of the mutants behaves as a competitor (dominant negative). Interference of homodimers of dysfunctional domains of Hoxb4 with the function of full-length Hoxb4 is not expected since (i) in preliminary short-term reconstitution experiments, detrimental effects on hematopoietic reconstitution were not seen with any of the (monomeric) deletion mutants (integrated proviruses were easily detected by Southern blot analysis in BM, spleen and thymus of primary recipients) and (ii) Hoxb4 does not homodimerize and cannot bind DNA as a homodimer. However, if One of these mutants (as a homo-dimer or a monomer) is problematic, different complementary mutants may be sought (which do not have dominant negative effects either as monomer of homodimers). The choice of these new complementary mutants may be based on the results of the (structure/function)studies mentioned above. Using the retrovirus, the relative expression levels of each mutant may also be changed (under a ribosomal reentry site or not). This may minimize the presence of deleterious homodimers and force the formation of heterodimers. Alternatively, if the formation of homodimers remain functionally problematic, the modified rapamycin system may be used.

Use of Retroviral Vectors to Express [FKBP12-Hoxb4 A+B] and Direct Protein Delivery of [TAT-FKBP12-Hoxb4 C−F] to Selectively Expand Retrovirally-Transduced HSC

In this experiment, retrovirally transduced HSC (which contain only one of the FKBP-Hoxb4 mutant) are exposed transiently to the complementary FKPB-Hoxb4 mutant through either direct protein delivery (TAT-fusion) or through adenoviral gene transfer.

This represents a dominant clonal selection system for HSC transduced with a retrovirus containing a dysfunctional Hoxb4 which should give a very significant (up to 55-fold under current conditions) expansion of retrovirally transduced stem cells. With this system, a retroviral gene transfer efficiency of 5% to primitive BM cells (as can be achieved with human BM cells) may translate to ˜75% of the reconstitution originating from retrovirally-transduced cells. In addition to obvious clinical possibilities, this system also represents an important tool to refine our understanding of the biology of Hoxb4 expressing HSC. The recent description of in vivo delivery of TAT proteins combined with the possibility of injecting FK1012 analogs to mice further increases the possibility to manipulate retrovirally-transduced HSC.

The above-mentioned examples improve our understanding of the molecular mechanisms utilized by the HOXB4 protein in order to expand HSC in a transplantation context in view of developing tools to manipulate the in vivo and in vitro expansion of these cells. Ultimately, these studies help identify partners and point to targets to Hoxb4. In addition, the findings derived from these studies help understand the normal mechanisms involved in the regulation of mouse and human HSC. Finally, the above examples clearly (indicate that the so-called “Hoxb4 effect” occurs very early after viral transduction, which may lead to clinical studies where Hoxb4 (or downstream effectors) could ultimately be utilized as a stem cell expanding (growth) factor.

### EXAMPLE VII

### In vitro Expansion of Hematopoietic Stem Calls by Recombinant TAT-HOXB4 Protein

Hematopoietic stem cells (HSCs) expand dramatically during fetal development and can self-renew extensively when transplanted in vivo. Conditions supporting significant in vitro HSC expansion are slowly being defined. We reported previously that retroviral over-expression of HOXB4 in murine bone arrow cells enables over 40-fold in vitro expansion of HSCs within <2 weeks. Based on these results, we have now engineered a recombinant TAT-HOXB4 protein as a potential growth factor for stem cells. HSCs exposed to 10-20 nM TAT-HOXB4 for 4 days expand by ˜4-6-fold over their input values and are 8-20-times more numerous than HSCs found in control cultures lacking this recombinant proteins. This level of expansion is comparable to that observed with retroviral (transduction of HOXB4 for a similar period of time. Moreover, the expanded stem cell population retains normal in vivo differentiating and long-term repopulating potentials. Our results also indicate that this growth-promoting effect of TAT-HOXB4 does not require accessory cells and predominantly targets primitive hematopoietic subpopulations. We thus demonstrate the feasibility of exploiting the potent growth-enhancing effects of an engineered HOXB4 soluble protein that enables rapid and significant ex vivo expansion of HSCs.

Generation of an Active Form of the TAT-HOXB4 Protein

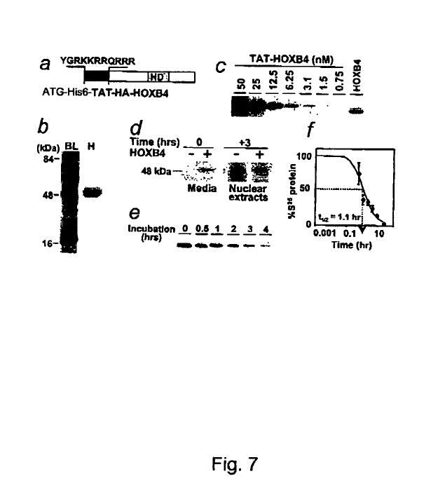

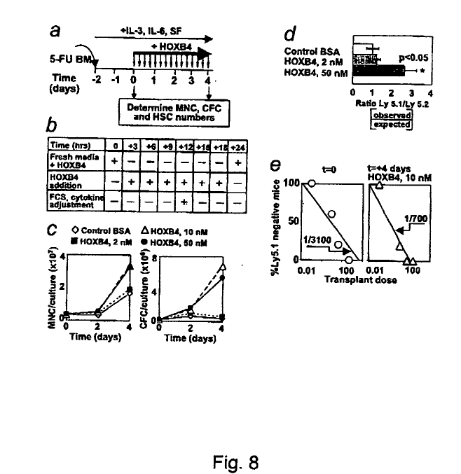

To test the possibility of achieving in vitro HSC expansion through direct HOXB4 protein delivery rather than by means of gene transfer, we elected to use recombinant TAT-HOXB4 fusion protein as depicted in FIGS. 7a-b. Preliminary experiments involving retrovirus-mediated gene transfer showed that the capacity of TAT-HOX4 to promote in the vitro expansion of clonogenic progenitors was similar to that of wild-type HOXB4. Since the magnitude of HSC expansion appears to correlate with the levels of available HOXB4 protein, attempts were made to identify concentrations of our soluble recombinant TAT-HOXB4 fusion protein (3-12 nM, see FIG. 7c) that would near the levels of HOX34 detected in hematopoietic cells engineered, by retroviral gene transfer, to overexpress this gene (FIG. 7c). Experiments performed with fibroblasts indicated that TAT-HOXB4 translocates rapidly from the media to nuclear compartments to achieve levels comparable to those detected in retrovirally-transduced cells (compare 4^{th} lane in FIG. 7d to 8^{th} lane in 7c). As TAT-fusion proteins distribute freely between the extra and intra-cellular compartments, it was critical to determine the half-life of HOXB4 in both compartments. The majority of TAT-HOXB4 was lost after 4 hours of incubation in medium with serum (FIG. 7e), and the half-life of intracellular HOXB4 determined by pulse chase experiments was—one hour (FIG. 7f). Based on these observations, we opted to introduce the. TAT-HOXB4 protein at every 3 hours in our cultures (see FIG. 8a).

The first set of experiments was performed with unpurified mouse bone marrow (BM) cells and was designed to test the biological activity and the range of TAT-HOXB4 concentrations required for HSC expansion. Modelled after our previous ex vivo HSC expansion studies, BM cells isolated from mice treated with 5-FU 4 days previously were first stimulated by growth factors for 2 days and then exposed to TAT-HOXB4 for 4 additional days (FIGS. 8a and b). Output of absolute numbers of HSCs as well as clonogenic myeloid (progenitors and total cells were determined (FIG. 8a)

At a 2 nM TAT-HOXB4, mononuclear cell (MNC) expansion was similar to control BM (see diamond vs gray squares in FIG. 8c). Modest (˜2-fold) but significant expansion of MNC was obtained when 10 or 50 nM of the protein was used (FIG. 8c, left). Similarly, clonogenic progenitor numbers (CFC) did not expand within the 4-day period when exposed to the 2 nM TAT-HOXB4, but expanded significantly in cultures at 10 nM and, a little less at 50 nM TAT-HOXB4 (FIG. 8c, right graph). The greater expansion of CFC compared to total mononuclear cells in response to optimal TAT-HOXB4 concentrations was significant (p<0.02) and in agreement with our previous observations which suggested that retrovirally-transduced HOXB4 exerts its largest proliferation-enhancing effect on more primitive hematopoietic cells.

TAT-HOXB4 and HSC Expansion

We next examined whether TAT-HOXB4 treatment affected the competitive repopulation capacity of treated HSCs in long-term transplantation experiments (18 wks). For these experiments, albumin-(control) or TAT-HOXB4-treated cells (Ly 5.1⁺) were grown in cultures as detailed in FIGS. 8a-b and competed at a ratio of 1:3 to 1:6 with competitor cells derived from a congenic mouse (Ly5.2⁺) similarly cultured as controls (i.e. without TAT-HOXB4). As expected, peripheral blood reconstitution of mice transplanted with the 1:3 combination of control+competitor cells maintained the initial 1:3 Ly5.1⁺/Ly5.2⁺ cell ratio when analysed at 18 wks post transplantation (FIG. 8d). Cells exposed to 2 n of TAT-HOXB4 were not more competitive than controls (gray bar, FIG. 8d) but higher concentrations of TAT-HOXB4 (50 nM) rendered the cells much more competent in reconstituting lymphoid and myeloid lineages as suggested by ratio of observed: expected reconstitution nearing the value of 3 (FIG. 8d).

To more accurately determine the effect of TAT-HOXB4 on HSC expansion, a pilot experiment was performed using the CRU assay. In this experiment, cells were transplanted in a limit dilution series at the beginning (tₒ) and end (t=+4 days) of exposure to 10 nM TAT-HOXB4 and HSC frequency determined based on the proportion of reconstituted animals 16-18 wks after transplantation. Using this assay, HSC frequency in starting (tₒ) cultures was 1/3100 (95% confidence interval=1/1100-1/7500), and increased to 1/700 (frequency adjusted to to: 95% confidence interval=1/300-1/2100) within the 4-day exposure to TAT-HOXB4 (FIG. 8e). This initial experiment demonstrated a net HSC expansion in culture conditions that are poorly supportive to HSCs (net loss are expected to occur in the absence of TAT-HOXB4).

TAT-HOXB4 Expands Purified HSCs without Affecting Differentiation

A second series of experiments (n=3) was performed using bone marrow populations enriched for HSC content based on expression of Sca-1 and absence of lineage-markers (so called Sca-130 Lin⁻ cells). These experiments were designed to assess whether the HSC-expanding activity of TAT-HOXB4 was direct, or whether it occurred through activation of mature accessory cells (e.g., macrophages, etc.), and to further compare the net HSC expansion in cultures containing TAT-HOXB4 versus controls (BSA or TAT-GFP).

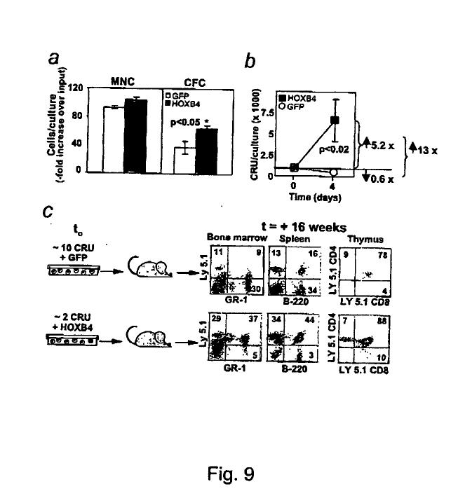

As observed for unpurified cells (FIG. 8c), the addition of TAT-HOXB4 had only a modest impact on the expansion MNC but a more important expansion was observed with colony-forming cells (CFC, FIG. 9a). In the first experiment, the numbers of HSCs in purified Sca-1⁺Lin⁻ populations were evaluated by limit dilution CRU assay right before the introduction of the TAT-proteins and determined at 1 in 40 (95% confidence interval: 1/25 to 1/69, Table 1). HSC numbers in Sca-1⁺Lin⁻ populations exposed to BSA (control) decreased within the 4-day culture to ˜50% of input values (from 2000 to 1100, see 5^{th} column, Table 1). In sharp Contrast, there was a net 4-fold increase in HSC numbers in cultures exposed to 20 nM TAT-HOXB4 (Table 1), for a 8-fold difference between BSA and TAT-HOXB4 treated populations. In this first experiment, reconstitution was determined based on lympho-myeloid reconstitution of peripheral blood.

TABLE 1
TAT-HOXB4 expands HSCs

| | | | CRU Frequency² | | | |
|---|---|---|---|---|---|---|
| | | Treat- | Peripheral Blood³ | | BM, Spleen, Thymus⁴ | |
| | Time¹ | ment | Freq. | Total | Freq. | Total |
| Expt: | Input, | none | 1/40 | 2000 | ND | ND |
| | Day 0 | | (1/25-1/69) | 2000 | ND | ND |
| | (tₒ) | | | | | |
| | Day +4 | BSA⁵ | 1/68 | 1100 | ND | ND |
| | | | (1/42-1/111) | | | |
| | Day +4 | HOXB4 | 1/14 | 8000 | ND | ND |
| | | | (1/8-1/22) | | | |
| Expt.I | Input, | none | 1/37 | 900 | 1/54 | 600 |
| | Day 0 | | (1/23-1/59) | | (1/38-1/133) | |
| | (tₒ) | | | | | |
| | Day +4 | GFP | 1/61 | 500 | 1/151 | 200 |
| | | | (1/37-1/101) | | (1/102-1/287C | 200 |
| | Day +4 | HOXB4 | 1/6 | 6000 | 1/9 | 4000 |
| | | | (1/3-1/10) | | (1/7-1/32) | |
| Expt.I | Day +4 | GFP | 1/87 | 400 | 1/160 | 200 |
| | | | (1/69-1/125) | | (1/96-1/220) | |
| | Day +4 | HOXB4 | 1/10 | 3000 | 1/16 | 2000 |
| | | | (1/7-1/18) | | (1/7-1/32) | |

¹As detemined in FIG. 8a
²CRU frequencies (95% C.I.) are expressed as tₒ equivalent and were determined at ≧16 weeks post transplant
³CRU analysis based on reconstitution of Ly5.2 recipients by lymphoid and myeloid peripheral blood Ly5.1⁺ cells.
⁴CRU analysis based on reconstitution of Ly5.2 recipients by BM-myeloid (Mac-1⁺) + spleen-lymphoid (B-220⁺) + thymus-lymphoid (CD4⁺CD8⁺) cells Ly5.1⁺cells.
⁵BSA, bovine serum albumin

Two additional experiments (Expt. II and III) were performed but this time TAT-GFP was introduced in control cultures instead of BSA and reconstitution evaluated following autopsy of all recipients sacrificed >16 wks in order to assess reconstitution of bone marrow myeloid (Mac-1⁺), spleen B cells (B220⁺) and thymic T cells (CD4 and CD8⁺). This provided a more rigorous evaluation of HSC which, be definition, should reconstitute all hemopoietic lineages for prolonged period of time (>12 wks). These experiments first indicated that TAT-GFP was similar to BSA, since both were ineffective in supporting HSC expansion over the 4-day culture. In experiment II, HSC frequency determined at tₒ was 1 in 54 (absolute 600 cells) and decreased to one third or 1 in 151 (200 absolute) after 4 days of culture in the presence of TAT-GFP. When the cells were exposed to TAT-HOXB4, a total of 4000 HSCs were present after the 4-day culture for a net difference of 20-fold over values determined for controls and representing a net 6-fold expansion over the input numbers (see last column in Table 1). Similar values were obtained in experiment III (Table I). The net and relative (to control) HSC expansion values obtained for all 3 experiments shown in Table 1 are summarized in FIG. 9b where the presence of TAT-HOXB4 led to a 5-fold net expansion in HSCs in 4 days with a 13-fold relative difference in HSC numbers when compared to controls.

The expanded HSCs exposed to TAT-HOXB4 were highly competitive and capable of multi-lineage differentiation. Reconstitution of representative recipients of 10 or 2 HSCs exposed for 4 days to TAT-GFP or TAT-HOXB4, respectively, are shown in FIG. 9c. TAT-HOXB4 treatment provided a much greater competitive advantage to 80 Sca-1⁺Lin⁻ cells (˜2 HSCs) than observed with as many as 400 of these cells exposed to TAT-GFP. Moreover, TAT-HOXB4-treated cells differentiated into all lineages analysed including all expected CD4 and CD8 populations in the thymus.

Together, these experiment show that TAT-HOXB4 stimulates the ex vivo expansion of fully competent HSCs Importantly, TAT-HOXB4 treatment does not increase the proliferation potential of treated HSCs, as recipients reconstituted with a single expanded HSC exhibited reconstitution levels comparable controls, and no difference in total numbers of progenitors between the two groups could be detected at any level of reconstitution (data not shown). In the future, it will be interesting to further refine the protocol with respect to the duration and frequency of TAT-HOXB4 treatment, to determine the potential added value of combining TAT-HOXB4 with some of the molecules recently reported to regulate self-renewal divisions of HSC such as FGF1, to expand the target cell range to human cord blood-derived HSCs, and eventually to other adult stem cells.

TAT-HOX Fusion Protein Purification

pTAT-HA-HOXB4 vector was generated by inserting a PCR fragment encompassing HOXB4 ORF flanked by engineered Nco I and EcoR I into Nco I-EcoR I sites of pTAT-HA, and the fidelity of reading frame was verified by sequencing. pTAT-HA-GFP vector was generously provided by Dr. S. F. Dowdy, Washington University School of Medicine, St. Louis, Mo. Purification of TAT fusion proteins was described. Briefly, the pTAT-HA-HOXB4- or pTAT-HA-GFP-transformed Bl21(DE3)pLysS cells (Novagen, Madison, Wis.) were induced for 2 hrs with 1 mM IPTG, and sonicated in buffer A (8M urea, 20 mM HEPES[pH 8.01], 100 mM NaCl). Lysates were clarified by centrifugation (20,000 rpm for 30 min at 20° C.), adjusted to 10 mM imidazole concentration, and loaded on HisTrap chelating columns. Bound proteins were eluted with 50, 100, and 250 mM imidazole in buffer A. TAT-HOXB4-containing fractions were loaded on MonoSP column in buffer B (4M urea, 20 mM HEPES[pH 6.5], 50 mM NaCl), eluted with 1 M NaCl, 20 mM HEPES, pH 8.0, and desalted on PD-10 Sephadex G-25. All separation columns used were obtained from Amersham Pharmacia, Piscataway, N.J. TAT-GFP was eluted from HisTrap columns with 250 mM imidazole in fractions with >95purity, and was directly subjected to desalting. Eluates (TAT-HOXB4 or TAT-GFP in PBS) were supplemented with BSA (0.5%) and glycerol (5%), aliquoted, and flash frozen at −80° C.

TAT-HOXB4 Transduction

BM cells were first cultured for 2 days in BM media (DMEM, 10% fetal calf serum [FCS], IL-3 [5 ng/mL], IL-6 [10 ng/mL], SF [100 ng/mL], Gentamycin [50 ug/mL] and Ciproxycin [10 ug/mL]), and then for 4 days in BM media containing TAT-HOXB4 (2-50 nM), or BSA (1%), or TAT-GFP (20 nM) (FIG. 8a). On day 3 (tₒ of treatment, FIG. 8a), cells (3×10⁵/mL) were resuspended in BM media supplemented with BSA, or TAT-GFP, or TAT-HOXB4. Fresh BSA or TAT fusion proteins (50% of the initial protein amount, in 5% of total culture media) were then added every 3 hrs. At +12 hrs, FSC and cytokines were added to correct for the resulting 20% dilution of culture media. At +24 hrs, cells resuspended in fresh BM media containing the protein of interest (FIG. 8b).

Mice and BM Transplantation

BM cells were obtained from (C57Bl/6Ly-Pep3b×C3H/HeJ)F1 mice 4 days after injection of 5-fluorouracil (5-FU, 150 mg/kg), and Sca⁺Lin⁻ subpopulations were purified as described. For limiting dilution experiments, different numbers of cells (2000-1×10⁶ for total BM, and 3-6000 Sca⁺Lin⁻ cells, 5-10 mice per group) were transplanted in lethally irradiated congenic recipients (C57Bl/6J×C3H/HeJ)F1, together with 1×10⁵ fresh BM cells. For competitive repopulation assays, transplantation inocula (1.5×10⁶ cells) comprised 30% of Ly 5.1 cells exposed to BSA, or 2 nM TAT-HOXB4, or 15% of cells exposed to 50 nM TAT-HOXB4, mixed with Ly 5.2 competitors that were not exposed to TAT-HOXB4, but were otherways treated exactly like the test cell populations.

Methylcellulose Cultures, Flow Cytometry and CRU Assay

On days 0, 2 and 4 of treatment, viable (trypan dye excluding) cells were counted, suitable aliquots were plated in standard methylcellulose, and colonies were scored on Day 10. Sca-1⁺Lin⁻ cells were isolated as described¹¹. To determine contribution of the transplanted Ly 5.1⁺ BM cells to reconstitution of myeloid and lymphoid compartments of transplantation chimeras, cells isolated from peripheral blood, or BM, spleens and thymi were stained with PE-conjugated anti Ly 5.1, FITC-conjugated antibodies recognizing Mac-1, GR-1, B-220, CD4, or allophycocyanin-conjugated CD8 as described and fractions of PE⁺(Ly 5.1) cells expressing a given cell surface antigen were determined by flow cytometry. HSC numbers in cultured BM populations were evaluated using a limiting dilution transplantation-based assay (CRU assay). Contributions of the transplanted Ly 5.1⁺ cells to peripheral blood MNC were determined at 16-20 weeks post transplant by flow cytometry as described above. To determine frequencies of cells capable of tri-lineage reconstitution, recipients were sacrificed at ≧6 weeks post-transplant, and proportions of Ly 5.1⁺ cells in their BM (myeloid, Mac-1), spleen (lymphoid, B-220) and thymus (CD4+CD8) determined as described above. For CRU determination from peripheral blood analysis, recipients >1% Ly 5.1⁺ cells in myeloid (Mac-1 or GR-1) and lymphoid (B-220, or B-220 and CD4+CD8) subpopulations were considered to be repopulated with at least 1 transplant derived CRU. CRU frequencies were calculated using Limit Dilution Analysis software (StemCell Technologies, Vancouver, BC).

Western Blotting and Determination of Intracellular HOXB4 Stability

Preparation of nuclear extracts and Western blotting were performed as described. Antibodies used were rat anti-HOXB4 (Developmental studies Hybridoma Bank, University of Iowa), and horseradish peroxidase-conjugated anti-rat antibody (Santa Cruz Biotech., Santa Cruz, Calif.). Pulse-chase experiments were performed as described. The total amount of radioactive proteins and the HOXB4 content at different time points were measured using STORM 860 and ImageQuant 5 software (Molecular Dynamics, Sunnyvale; Calif.). Half-life of HOXB4 was calculated using AllFit (©Charles and Andree Lean, University of Montreal, QC).

While the invention has been described in connection with specific embodiments thereof, it were understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A stem cell expansion factor, which comprises an amino acid sequence having the expansion enhancement activity of a peptide encoded by a Hox nucleotide sequence enhancing expansion of a stem cell population, and wherein said amino acid sequence is able to cross a cell membrane.

2. The factor according to claim 1, wherein said amino acid sequence consists of a HOXB4 protein.

3. The factor according to claim 1, wherein said amino acid sequence further comprises an HIV-derived peptide able to cross a cell membrane.

4. The factor according to claim 3, wherein said HIV-derived peptide consists of a NH₂-terminal protein transduction domain (PTD) from a transactivating protein (TAT).

5. The factor according to claim 4, wherein said stem cell is a hematopoietic stem cell.

6. The factor according to claim 5, wherein said hematopoietic stem cell is human or mouse.

7. A method for enhancing expansion of a stem cell population, the method comprising directly delivering in a stem cell population an effective amount of the stem cell expansion factor of claim 1, whereby said amino acid sequence is able to cross a cell membrane and is substantially active in said stem cell population, thereby enhancing expansion of said stem cell population.

8. A method according to claim 7, wherein said amino acid sequence consists of a HOXB4 protein.

9. A method according to claim 7, wherein the amino acid sequence is delivered in said stem cell population in vivo.

10. A method according to claim 7, wherein said amino acid sequence further comprises an HIV-derived peptide able to cross a cell membrane.

11. A method according to claim 10, wherein said HIV-derived peptide consists of a NH₂-terminal protein transduction domain (PTD) from a transactivating protein (TAT).

12. A method according to claim 11, wherein said stem cell is a hematopoietic stem cell.

13. A method according to claim 12, wherein said hematopoietic stem cell is human.

14. A drug-inducible method for enhancing expansion of a stem cell population, the method comprising:
a) delivering in a stem cell population a nucleotide sequence linked to a drug-binding protein and encoding one of a DNA-binding domain and a NH₂₋terminal domain of a peptide having the activity of a Hox protein able to enhance expansion of said stem cell population, delivering in said stem cell population a nucleotide sequence encoding the remainder of the DNA-binding domain and N-terminal domains linked to a drug-binding protein; and
b) exposing said stem cell to a dimerizing agent;
whereby a functionally active protein is reconstituted in said stem cell population, thereby enhancing expansion of said stem cell.

15. A method according to claim 14, wherein said drug-binding protein consists of FKBP12, and wherein said dimerizing agent consists of FK1012 or an analog thereof.

16. A method according to claim 15, wherein said stem cell is a hematopoietic stem cell.

17. A method according to claim 16, wherein said hematopoietic stem cell is human.

18. A method for restoring a patient hematopoietic capability, said method comprising directly delivering in a hematopoietic stem cell population of a patient the stem cell expansion factor of claim 1, wherein said amino acid sequence is able to cross a cell membrane and is substantially active in said hematopoietic stem cell, thereby enhancing expansion of said hematopoietic stem cell population and restoring hematopoietic capability of said patient.

19. A method according to claim is, wherein said amino acid sequence consists of a HOXB4 peptide.

20. A method according to claim 1, wherein said amino acid sequence is delivered in said hematopoietic stem cell in vivo.

21. A method according to claim 18, wherein said amino acid sequence further comprises an HIV-derived peptide able to cross a cell membrane.

22. A method according to claim 21, wherein said HIV-derived peptide consists of a NH₂-terminal protein transduction domain (PTD) from a transactivating protein (TAT).

23. A method according to claim 19, wherein said hematopoietic stem cell is human.

24. A composition comprising a hematopoietic stem cell population having enhanced expansion capability, said hematopoietic stem cell population being generated by directly delivering therein the stem cell expansion factor of claim 1 and which is functionally active therein, in association with a pharmaceutically acceptable carrier.
